(19) United States
(12) Patent Application Publication
(10) Pub. No.: US 2023/0165204 A1
(43) Pub. Date: Jun. 1, 2023

(54) METHODS AND SYSTEMS FOR USING ENVIROTYPE IN GENOMIC SELECTION

(71) Applicant: Inari Agriculture Technology, Inc., Cambridge, MA (US)

(72) Inventors: Maria Elena FARICELLI, Newton, MA (US); Keru CHEN, Arlington, MA (US)

(73) Assignee: Inari Agriculture Technology, Inc., Cambridge, MA (US)

(21) Appl. No.: 17/920,741

(22) PCT Filed: Apr. 22, 2021
(86) PCT No.: PCT/US2021/028649

§ 371 Date: Oct. 21, 2022

Related U.S. Application Data
(60) Provisional application No. 63/014,641, filed on Apr. 23, 2020.

(51) Int. Cl. A01H 1/04

(52) U.S. Cl. CPC A01H 1/04

(57) ABSTRACT

Provided herein are methods for using envirotype in genomic prediction, genomic selection, variety development, and breeding. Also provided herein are systems for implementing such methods, as well as computer-readable storage media storing instructions for performing such methods.

### CROSS-REFERENCE TO RELATED APPLICATION

This application claims priority to U.S. Provisional Pat. Application No. 63/014,641 filed on Apr. 23, 2020, the entirety of which is incorporated herein by reference.

### FIELD

The present disclosure relates generally to the field of genetics and breeding, and more specifically to methods and systems for using envirotype information in genomic selection.

### BACKGROUND

Conventional breeding relies largely on phenotypic evaluation through cycles of crossing and selection, which requires substantial breeding efforts with over multiple years to develop an improved variety. The major challenge lies in the low efficiency of phenotypic selection for desirable traits of a quantitative nature that are controlled by many genes of small effects. Thus, efficient methods have been searched to improve the selection of individual plants with desired traits. Marker-assisted selection (MAS) is based on the selection of statistically significant genetic marker-trait associations in conventional breeding programs without observing phenotypic variation in the traits. However, traditional MAS is not well suited for selecting complex traits controlled by many genes, for example, yield performance in maize.

More recently, genomic selection (GS) has emerged as a promising approach for efficient plant and animal breeding, which is a method of selection based on predicted genetic values of untested lines by using genome-wide marker information. In essence, a set of individuals that is both phenotyped and genotyped (“the training set”) is used to train a statistical model that is applied to predict unobserved individuals (“the prediction set”) on the basis of only genotyping data from the latter. GS has been shown to facilitate rapid selection of superior genotypes and, as a result, accelerate the breeding cycle. A shortcoming of genomic selection, however, is the accuracy of the prediction, which may be affected by various factors, including environmental effects. For instance, breeders’ mission to identify elite varieties across multiple environments, such as testing locations and years, is challenged by the known “genotype by environment” (GxE) interaction.

Accordingly, there is a need for new methods and systems of genomic selection with improved prediction accuracy. Such improved methods and systems can be useful for various applications, such as variety development and breeding of agricultural species.

### BRIEF SUMMARY

Provided herein are methods for using envirotype in genomic selection and breeding. Also provided herein are systems for implementing such methods, as well as computer-readable storage media storing instructions for performing such methods.

In one aspect, provided herein is a method for predicting phenotype data of a population in a geographic area, including: providing a first population of individuals in a first geographic area; obtaining genotype data, phenotype data, and envirotype data of the first population in the first geographic area; building a statistical model by associating the phenotype data of the first population with the genotype data and envirotype data of the first population; providing a second population of individuals in a second geographic area; obtaining genotype data and envirotype data of the second population in the second geographic area; and predicting phenotype data of the second population in the second geographic area by applying the statistical model to the genotype data and envirotype data of the second population. In some embodiments, the method further includes selecting one or more individuals from the second population based on the predicted phenotype data of the second population.

In another aspect, provided herein is a method of genomic selection, including: providing a first population of individuals in a first geographic area; obtaining genome-wide genotype data, phenotype data, and envirotype data of the first population in the first geographic area; building a statistical model by associating the phenotype data of the first population with the genome-wide genotype data and envirotype data of the first population: providing a second population of individuals in a second geographic area; obtaining genome-wide genotype data and envirotype data of the second population in the second geographic area; predicting phenotype data of the second population in the second geographic area by applying the statistical model to the genome-wide genotype data and envirotype data of the second population; and selecting one or more individuals from the second population based on the predicted phenotype data of the second population.

In yet another aspect, provided herein is a method for developing one or more varieties suitable for a geographic area, including: providing a first population of individuals in a first geographic area; obtaining genotype data, phenotype data, and envirotype data of the first population in the first geographic area; building a statistical model by associating the phenotype data of the first population with the genotype data and envirotype data of the first population; providing a second population of individuals in a second geographic area; obtaining genotype data and envirotype data of the second population in the second geographic area; predicting phenotype data of the second population in the second geographic area by applying the statistical model to the genotype data and envirotype data of the second population; selecting one or more individuals from the second population based on the predicted phenotype data of the second population; and developing one or more varieties from the selected one or more individuals, wherein the one or more varieties exhibit suitable phenotype for the second geographic area.

In still another aspect, provided herein is a method of breeding, including: providing a first population of individuals in a first geographic area; obtaining genotype data, phenotype data, and envirotype data of the firsₜ population in the first geographic area; building a statistical model by associating the phenotype data of the first population with the genotype data and envirotype data of the first population; providing a second population of individuals in a second geographic area; obtaining genotype data and envirotype data of the second population in the second geographic area; predicting phenotype data of the second population in the second geographic area by applying the statistical model to the genotype data and envirotype data of the second population: selecting one or more individuals from the second population based on the predicted phenotype data of the second population; and using the selected one or more individuals in breeding.

In some embodiments, the individuals in the first population are inbred lines, breeding populations, or hybrids, and the individuals in the second population are segregating lines from breeding populations. In some embodiments, the individuals in the first population are hybrids, and the individuals in the second population are inbred lines and hybrids that may or may not have parental inbred lines in common with the hybrids from the first population. In some embodiments, the individuals in the first population are parental lines and the individuals in the second population are filial lines derived from the parental lines.

In some embodiments, the selection is for advancing the selected one or more individuals to a further stage in a breeding program. In some embodiments, the selection is for testing performance of the selected one or more individuals in a field. In some embodiments, the selected one or more individuals are segregating lines, inbred lines, or hybrid lines. In some embodiments, the selection is applied using a selection intensity.

In some embodiments, the method further includes producing offspring from the selected one or more individuals. In some embodiments, the offspring are produced by selfing, crossing, or asexual propagation. In some embodiments, the method further includes growing the offspring into maturity.

In some embodiments that may be combined with any of the preceding embodiments, the first population is a training population and the second population is a prediction population. In some embodiments, the second population is a genetically diverse population. In some embodiments, the second population is a uniform population. In some embodiments, the second population is an individual.

In some embodiments that may be combined with any of the preceding embodiments, the first geographic area and the second geographic area are the same geographic area. In some embodiments, the second geographic area is a target geographic area.

In some embodiments that may be combined with any of the preceding embodiments, the envirotype data is time data, location data, weather data, soil data, companion organism data, management data, crop canopy data, cultivation area data, or a combination thereof. In some embodiments, the time data is century, decade, year, season, month, day, hour, minute, second, or a combination thereof. In some embodiments, the location data is latitude, longitude, altitude, or a combination thereof. In some embodiments, the weather data is temperature, humidity, pressure, zonal wind speed, meridional wind speed, long-wave radiation, fraction of total precipitation that is convective, convective available potential energy, potential evaporation, precipitation hourly total, short-wave solar radiation, photoperiod, or a combination thereof. In some embodiments, the soil data is soil type, soil structure, soil moisture, soil depth, soil organic matter content, soil density, soil pH, soil fertility, soil salinity, or a combination thereof. In some embodiments, the companion organism data is soil fauna, insects, animals, weeds, or a combination thereof. In some embodiments, the management data is intercropping management, cover-cropping management, rotating cropping management, or a combination thereof. In some embodiments, the crop canopy data is obtained from an aerial platform. In some embodiments, the envirotype data is grouped according to the growth stages of the individuals. In some embodiments, the envirotype data is an envirotype map.

In some embodiments that may be combined with any of the preceding embodiments, the one or more individuals are a crop selected from the group consisting of maize, soybean, wheat, sorghum, barley, oats, rice, millet, canola, cotton, cassava, cowpea, safflower, sesame, tobacco, flax, sunflower, a grain crop, a vegetable crop, an oil crop, a forage crop, an industrial crop, a woody crop, and a biomass crop.

In some embodiments that may be combined with any of the preceding embodiments, the statistical model estimates the effects of genetic markers in interactions with the envirotype on the phenotype of the individuals of the first population. In some embodiments, the statistical model includes a genotype variable, an envirotype covariate, and an interaction term between the genotype variable and the envirotype covariate. In some embodiments, the statistical model is a linear regression model, a logistic regression model, a Bayesian ridge regression model, a lasso regression model, an elastic net regression model, a decision tree model, a gradient boosted tree model, a neural network model, or a support vector machine model. In some embodiments, the predicted phenotype data of the second population are genomic estimated breeding values (GEBVs). In some embodiments, building the statistical model further includes training the statistical model, tuning the statistical model, validating the statistical model, and/or updating the statistical model.

In certain aspect, the present invention provides a variety developed by any one of the preceding methods.

In still another aspect, provided herein is a computer-implemented method for predicting phenotype data of a population in a geographic area, including: receiving a dataset including: genotype data, phenotype data, and envirotype data of a first population of individuals in a first geographic area, and genotype data and envirotype data of a second population of individuals in a second geographic area; and performing a prediction of phenotype data of the second population in the second geographic area, by applying a statistical model to the genotype data and envirotype data of the second population, wherein the statistical model is obtained by associating the phenotype data of the firsₜ population with the genotype data and envirotype data of the first population in the first geographic area. In some embodiments, the method further includes selecting one or more individuals from the second population based on the predicted phenotype data of the second population. In some embodiments, the statistical model is a linear regression model, a logistic regression model, a Bayesian ridge regression model, a lasso regression model, an elastic net regression model, a decision tree model, a gradient boosted tree model, a neural network model, or a support vector machine model.

In still another aspect, provided herein is a computer-readable storage medium storing computer-executable instructions, including: instructions for building a statistical model from a first dataset, wherein the dataset includes genotype data, phenotype data, and envirotype data of a first population of individuals in a first geographic area, wherein the statistical model associates the phenotype data of the first population with the genotype data and envirotype data of the firsₜ population in the first geographic area; instructions for applying the statistical model to a second dataset, wherein the second dataset includes genotype data and envirotype data of a second population of individuals in a second geographic area; and instructions for calculating estimated phenotype data of the second population from application of the statistical model to the second dataset. In some embodiments, the computer-readable storage medium further includes instructions for selecting one or more individuals from the second population based on the estimated phenotype data of the second population. In some embodiments, the estimated phenotype data of the second population are genomic estimated breeding values (GEBVs).

In still another aspect, provided herein is a system for estimating phenotype data of a population in a geographic area, including: a computer-readable storage medium storing a database including: genotype data, phenotype data, and envirotype data of a first population of individuals in a first geographic area, and genotype data and envirotype data of a second population of individuals in a second geographic area; a computer-readable storage medium storing computer-executable instructions, including: instructions for building a statistical model from associating the phenotype data of the first population with the genotype data and envirotype data of the first population in the first geographic area; instructions for applying the statistical model to the genotype data and envirotype data of the second population in the second geographic area; and instructions for calculating estimated phenotype data of the second population from application of the statistical model to the genotype data and envirotype data of the second population in the second geographic area; and a processor configured to execute the computer-executable instructions stored in the computer-readable storage medium. In some embodiments, the computer-readable storage medium further includes instructions for selecting one or more individuals from the second population based on the estimated phenotype data of the second population. In some embodiments, the statistical model is a linear regression model, a logistic regression model, a Bayesian ridge regression model, a lasso regression model, an elastic net regression model, a decision tree model, a gradient boosted tree model, a neural network model, or a support vector machine model. In some embodiments, the estimated phenotype data of the second population are genomic estimated breeding values (GEBVs).

In one aspect, provided herein is a method of breeding, including: providing a first population of individuals in a first geographic area; obtaining genotype data, phenotype data, and envirotype data of the first population in the first geographic area; building a statistical model by associating the phenotype data of the first population with the genotype data and envirotype data of the firsₜ population; providing a second population of individuals in a second geographic area; obtaining genotype data and envirotype data of the second population in the second geographic area; predicting phenotype data of the second population in the second geographic area by applying the statistical model to the genotype data and envirotype data of the second population; selecting one or more individuals from the second population based on the predicted phenotype data of the second population; and using the selected one or more individuals in breeding.

In another aspect, provided herein is a method for predicting phenotype data of a population in a geographic area for use in breeding, including: providing a first population of individuals in a first geographic area; obtaining genotype data, phenotype data, and envirotype data of the first population in the first geographic area; building a statistical model by associating the phenotype data of the first population with the genotype data and envirotype data of the first population; providing a second population of individuals in a second geographic area; obtaining genotype data and envirotype data of the second population in the second geographic area; and predicting phenotype data of the second population in the second geographic area by applying the statistical model to the genotype data and envirotype data of the second population. In some embodiments, the method further includes selecting one or more individuals from the second population based on the predicted phenotype data of the second population. In some embodiments, the method further comprises selecting one or more individuals from the second population based on the predicted phenotype data of the second population; and using the selected one or more individuals in breeding.

In another aspect, provided herein is a method of genomic selection, including: providing a first population of individuals in a first geographic area; obtaining genome-wide genotype data, phenotype data, and envirotype data of the first population in the first geographic area; building a statistical model by associating the phenotype data of the first population with the genome-wide genotype data and envirotype data of the first population; providing a second population of individuals in a second geographic area; obtaining genome-wide genotype data and envirotype data of the second population in the second geographic area; predicting phenotype data of the second population in the second geographic area by applying the statistical model to the genome-wide genotype data and envirotype data of the second population; and selecting one or more individuals from the second population based on the predicted phenotype data of the second population. In some embodiments, the method further comprises using the selected one or more individuals in breeding.

In yet another aspect, provided herein is a method for developing one or more varieties suitable for a geographic area, including: providing a first population of individuals in a first geographic area: obtaining genotype data, phenotype data, and envirotype data of the first population in the first geographic area; building a statistical model by associating the phenotype data of the first population with the genotype data and envirotype data of the first population; providing a second population of individuals in a second geographic area; obtaining genotype data and envirotype data of the second population in the second geographic area; predicting phenotype data of the second population in the second geographic area by applying the statistical model to the genotype data and envirotype data of the second population: selecting one or more individuals from the second population based on the predicted phenotype data of the second population; and developing one or more varieties from the selected one or more individuals, wherein the one or more varieties exhibit suitable phenotype for the second geographic area.

In some embodiments, the individuals in the first population are inbred lines, breeding populations, or hybrids, and the individuals in the second population are segregating lines from breeding populations. In some embodiments, the individuals in the first population are hybrids, and the individuals in the second population are inbred lines and hybrids that may or may not have parental inbred lines in common with the hybrids from the first population. In some embodiments, the individuals in the first population are parental lines and the individuals in the second population are filial lines derived from the parental lines.

In some embodiments, the selection is for advancing the selected one or more individuals to a further stage in a breeding program. In some embodiments, the selection is for testing performance of the selected one or more individuals in a field. In some embodiments, the selected one or more individuals are segregating lines, inbred lines, or hybrid lines. In some embodiments, the selection is applied using a selection intensity.

In some embodiments, the method further includes producing offspring from the selected one or more individuals. In some embodiments, the offspring are produced by selfing, crossing, or asexual propagation. In some embodiments, the method further includes growing the offspring into maturity.

In some embodiments that may be combined with any of the preceding embodiments, the first population is a training population and the second population is a prediction population. In some embodiments, the second population is a genetically diverse population. In some embodiments, the second population is a uniform population. In some embodiments, the second population is an individual.

In some embodiments that may be combined with any of the preceding embodiments, the first geographic area and the second geographic area are the same geographic area. In some embodiments, the second geographic area is a target geographic area.

In some embodiments that may be combined with any of the preceding embodiments, the envirotype data is time data, location data, weather data, soil data, companion organism data, management data, crop canopy data, cultivation area data, or a combination thereof. In some embodiments, the time data is century, decade, year, season, month, day, hour, minute, second, or a combination thereof. In some embodiments, the location data is latitude, longitude, altitude, or a combination thereof. In some embodiments, the weather data is temperature, humidity, pressure, zonal wind speed, meridional wind speed, long-wave radiation, fraction of total precipitation that is convective, convective available potential energy, potential evaporation, precipitation hourly total, short-wave solar radiation, photoperiod, or a combination thereof. In some embodiments, the soil data is soil type, soil structure, soil moisture, soil depth, soil organic matter content, soil density, soil pH, soil fertility, soil salinity, or a combination thereof. In some embodiments, the companion organism data is soil fauna, insects, animals, weeds, or a combination thereof. In some embodiments, the management data is intercropping management, cover-cropping management, rotating cropping management, or a combination thereof. In some embodiments, the crop canopy data is obtained from an aerial platform. In some embodiments, the envirotype data is grouped according to the growth stages of the individuals. In some embodiments, the envirotype data is an envirotype map.

In some embodiments that may be combined with any of the preceding embodiments, the one or more individuals are a crop selected from the group consisting of maize, soybean, wheat, sorghum, barley, oats, rice, millet, canola, cotton, cassava, cowpea, safflower, sesame, tobacco, flax, sunflower, a grain crop, a vegetable crop, an oil crop, a forage crop, an industrial crop, a woody crop, and a biomass crop.

In some embodiments that may be combined with any of the preceding embodiments, the statistical model estimates the effects of genetic markers in interactions with the envirotype on the phenotype of the individuals of the first population. In some embodiments, the statistical model includes a genotype variable, an envirotype covariate, and an interaction term between the genotype variable and the envirotype covariate. In some embodiments, the statistical model is a linear regression model, a logistic regression model, a Bayesian ridge regression model, a lasso regression model, an elastic net regression model, a decision tree model, a gradient boosted tree model, a neural network model, or a support vector machine model. In some embodiments, the predicted phenotype data of the second population are genomic estimated breeding values (GEBVs). In some embodiments, building the statistical model further includes training the statistical model, tuning the statistical model, validating the statistical model, and/or updating the statistical model.

In certain aspect, the present invention provides a variety developed by any one of the preceding methods.

In still another aspect, provided herein is a computer-implemented method for predicting phenotype data of a population in a geographic area for use in breeding, including: receiving genotype data and envirotype data of a population of individuals in a geographic area; applying a statistical model to the genotype data and envirotype data of the population to obtain a prediction of phenotype data of the population in the geographic area, wherein the statistical model is configured to receive genotype data and envirotype data of a population of individuals in a geographic area and output a prediction of phenotype data of the population in the geographic area; and outputting the prediction of phenotype data of the population in the geographic area. In some embodiments, the method further includes selecting one or more individuals from the population based on the predicted phenotype data of the population; and informing a user of the selected one or more individuals for breeding. In some embodiments, the statistical model is a trained model selected from the group consisting of linear regression model, a logistic regression model, a Bayesian ridge regression model, a lasso regression model, an elastic net regression model, a decision tree model, a gradient boosted tree model, a neural network model, and a support vector machine model.

In still another aspect, provided herein is a computer-readable storage medium storing one or more programs for predicting phenotype data of a population in a geographic area for use in breeding, the one or more programs comprising instructions, which when executed by one or more processors of an electronic device having a display, cause the electronic device to: receiving genotype data and envirotype data of a population of individuals in a geographic area; applying a statistical model to the genotype data and envirotype data of the population to obtain a prediction of phenotype data of the population in the geographic area, wherein the statistical model is configured to receive genotype data and envirotype data of a population of individuals in a geographic area and output a prediction of phenotype data of the population in the geographic area; and outputting the prediction of phenotype data of the population in the geographic area. In some embodiments, the computer-readable storage medium further includes instructions for selecting one or more individuals from the population based on the predicted phenotype data of the population; and informing a user of the selected one or more individuals for breeding. In some embodiments, the statistical model is a trained model selected from the group consisting of linear regression model, a logistic regression model, a Bayesian ridge regression model, a lasso regression model, an elastic net regression model, a decision tree model, a gradient boosted tree model, a neural network model, and a support vector machine model. In some embodiments, the estimated phenotype data of the population are genomic estimated breeding values (GEBVs).

In still another aspect, provided herein is an electronic device for predicting phenotype data of a population in a geographic area for use in breeding, comprising: a display; one or more processors; a memory; and one or more programs, wherein the one or more programs are stored in the memory and configured to be executed by the one or more processors, the one or more programs including instructions for: receiving genotype data and envirotype data of a population of individuals in a geographic area; applying a statistical model to the genotype data and envirotype data of the population to obtain a prediction of phenotype data of the population in the geographic area, wherein the statistical model is configured to receive genotype data and envirotype data of a population of individuals in a geographic area and output a prediction of phenotype data of the population in the geographic area; and outputting the prediction of phenotype data of the population in the geographic area. In some embodiments, the computer-readable storage medium further comprises instructions for selecting one or more individuals from the population based on the predicted phenotype data of the population; and informing a user of the selected one or more individuals for breeding. In some embodiments, the statistical model is a trained model selected from the group consisting of linear regression model, a logistic regression model, a Bayesian ridge regression model, a lasso regression model, an elastic net regression model, a decision tree model, a gradient boosted tree model, a neural network model, and a support vector machine model. In some embodiments, the predicted phenotype data of the population are genomic estimated breeding values (GEBVs).

### DESCRIPTION OF THE FIGURES

For a better understanding of the various described embodiments, reference may be made to the detailed description and examples below, in conjunction with the following drawings in which the reference numerals refer to corresponding parts throughout the figures.

FIG. 1 depicts a block diagram of an exemplary method for predicting phenotype data of a population in a geographic area.

FIG. 2 depicts a block diagram of an exemplary method of genomic selection.

FIG. 3 depicts a block diagram of an exemplary method for for developing one or more varieties suitable for a geographic area.

FIG. 4 depicts a block diagram of an exemplary method of breeding.

FIG. 5 depicts a block diagram of an exemplary computer-implemented method for predicting phenotype data of a population in a geographic area.

FIG. 6 depicts an exemplary electronic device in accordance with some embodiments.

### DETAILED DESCRIPTION

The following description is presented to enable a person of ordinary skill in the art to make and use the various embodiments. Descriptions of specific devices, techniques, and applications are provided only as examples. Various modifications to the examples described herein will be readily apparent to those of ordinary skill in the art, and the general principles defined herein may be applied to other examples and applications without departing from the spirit and scope of the various embodiments. Thus, the various embodiments are not intended to be limited to the examples described herein and shown, but are to be accorded the scope consistent with the claims.

Although the following description uses terms “first”, “second”, etc. to describe various elements, these elements should not be limited by the terms. These terms are only used to distinguish one element from another. For example, a first graphical representation could be termed a second graphical representation, and, similarly, a second graphical representation could be termed a first graphical representation, without departing from the scope of the various described embodiments. The first graphical representation and the second graphical representation are both graphical representations, but they are not the same graphical representation.

The terminology used in the description of the various described embodiments herein is for the purpose of describing particular embodiments only and is not intended to be limiting. As used in the description of the various described embodiments and the appended claims, the singular forms “a”, “an”, and “the” are intended to include the plural forms as well, unless the context clearly indicates otherwise. It will also be understood that the term “and/or” as used herein refers to and encompasses any and all possible combinations of one or more of the associated listed items. It will be further understood that the terms “includes”, “including”, “comprises”, and/or “comprising”, when used in this specification, specify the presence of stated features, integers, steps, operations, elements, and/or components, but do not preclude the presence or addition of one or more other features, integers, steps, operations, elements, components, and/or groups thereof.

The term “if” is, optionally, construed to mean “when” or “upon” or “in response to determining” or “in response to detecting”, depending on the context. Similarly, the phrase “if it is determined” or “if [a stated condition or event] is detected” is, optionally, construed to mean “upon determining” or “in response to determining” or “upon detecting [the stated condition or event]” or “in response to detecting [the stated condition or event]”, depending on the context.

The following description sets forth exemplary methods, parameters, and the like. It should be recognized, however, that such description is not intended as a limitation on the scope of the present disclosure but is instead provided as a description of exemplary embodiments.

Although the following description uses terms “first”, “second”, etc. to describe various elements, these elements should not be limited by the terms. These terms are only used to distinguish one element from another. For example, a first graphical representation could be termed a second graphical representation, and, similarly, a second graphical representation could be termed a first graphical representation, without departing from the scope of the various described embodiments. The first graphical representation and the second graphical representation are both graphical representations, but they are not the same graphical representation.

The terminology used in the description of the various described embodiments herein is for the purposes of describing particular embodiments only and is not intended to be limiting. As used in the description of the various described embodiments and the appended claims, the singular forms “a”, “an”, and “the” are intended to include the plural forms as well, unless the context clearly indicates otherwise. It will also be understood that the term “and/or” as used herein refers to and encompasses any and all possible combinations of one or more of the associated listed items. It will be further understood that the terms “includes”, “including”, “comprises”, and/or “comprising”, when used in this specification, specify the presence of stated features, integers, steps, operations, elements, and/or components, but do not preclude the presence or addition of one or more other features, integers, steps, operations, elements, components, and/or groups thereof.

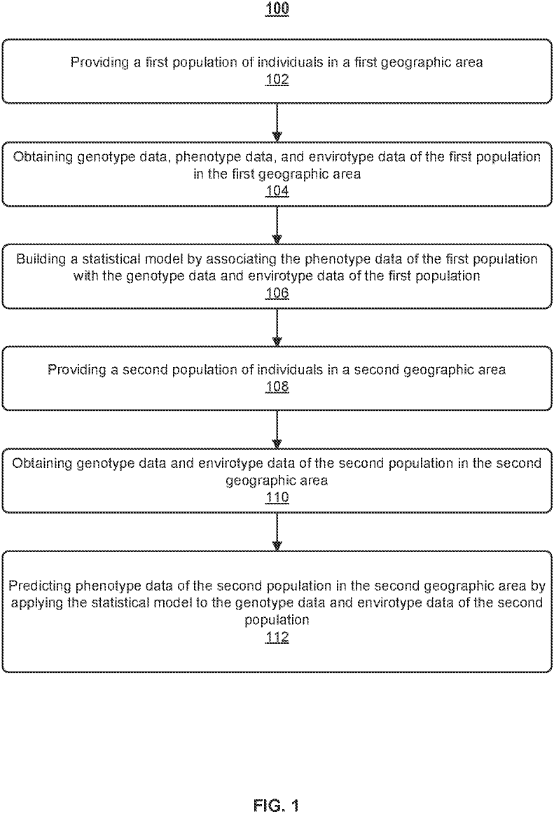

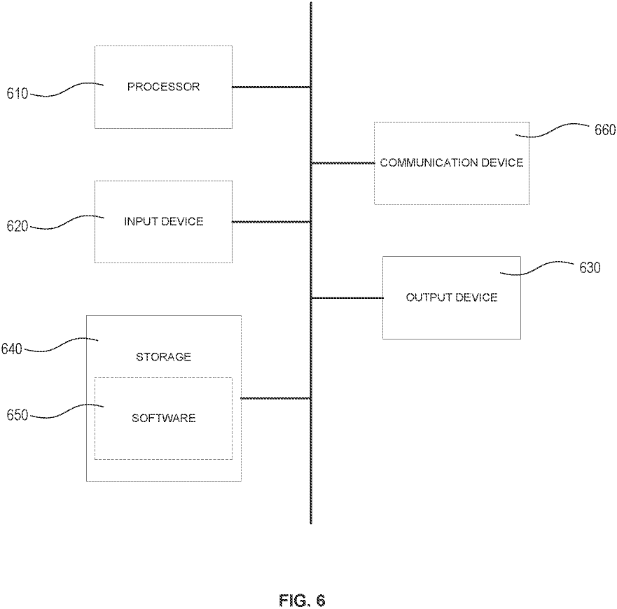

The present invention is based, in part, on the surprising results that increased effectiveness and efficiency of genomic selection are achieved by incorporating envirotype information into genomic selection models. Provided herein are methods for using envirotype in genomic prediction, genomic selection, variety development, and breeding, as depicted in FIGS. 1-5. Also provided herein are computer-implemented methods and systems for implementing such methods, as well as computer-readable storage media storing instructions for performing such methods. FIG. 6 illustrates an exemplary electronic device having a described computer system in accordance with some embodiments.

### Breeding for a Geographic Area

A major goal of agricultural breeding is to genetically improve the quality, diversity, and performance of agricultural species. It is important to note, however, that growth and development of crops and animals are heavily influenced by their surrounding environment. As a result, the geographic area in which breeding selection and testing take place can significantly affect the objectives and outcome of a breeding program. For instance, there is often a need to establish a breeding program in a specific geographic location in order to produce new varieties suitable for the specific area (“breeding zone”), e.g., a heat-tolerant cattle variety for a tropical region, or varieties that have certain desirable characteristics that cater to local consumers’ preference in the product market (“market zone”), e.g., a white-kernel corn variety that is preferred in Mexico. Additionally, expression of a trait, such as yield, can be largely dependent on the management, control, and improvement of the environment where the species grows, rendering its selection and testing sensitive to environmental variation.

Accordingly, in one aspect, provided herein is a method for predicting phenotype data of a population in a geographic area, including: providing a first population of individuals in a first geographic area; obtaining genotype data, phenotype data, and envirotype data of the first population in the first geographic area; building a statistical model by associating the phenotype data of the first population with the genotype data and envirotype data of the first population; providing a second population of individuals in a second geographic area; obtaining genotype data and envirotype data of the second population in the second geographic area; and predicting phenotype data of the second population in the second geographic area by applying the statistical model to the genotype data and envirotype data of the second population.

As used herein, the term “first geographic area” refers to a geographic area for the purposes of training or building a statistical model. The first geographic area may include various suitable envirotypes. Examples of envirotypes are provided below in the “Envirotype” section. In some embodiments, the first geographic area contains a plurality of distinct envirotypes.

As used herein, the term “second geographic area” refers to a geographic area for the purposes of predicting phenotype data. The second geographic area may include various suitable envirotypes. Examples of envirotypes are provided below in the “Envirotype” section. In some embodiments, the second geographic area contains a plurality of distinct envirotypes.

The first geographic area and the second geographic area may or may not be the same geographic area. In some embodiments, the first geographic area and the second geographic area are different but overlapping geographic areas. In some embodiments, the second geographic area is a subset of the first geographic area.

With reference to FIG. 1, the first geographic area in 102 and the second geographic area in 108 may be the same geographic area in some examples, and may be different geographic areas in some other examples. In some embodiments, the second geographic area in 108 is a target breeding zone. In some embodiments, the second geographic area in 108 is a target market zone. In some embodiments, the method further includes selecting one or more individuals from the second population based on the predicted phenotype data of the second population after the step 112.

### Genomic Prediction and Selection

Genomic selection (GS, see e.g., Goddard et al, 2009) aims to use genome-wide markers to estimate the effects of all loci affecting a trait and thereby compute a genomic estimated breeding value (GEBV), achieving more comprehensive and reliable selection than marker assisted selection (MAS). MAS, a strategy commonly used in plant molecular breeding, is suitable only for traits controlled by a small number of major genes (see e.g., Lande et al, 1990). However, most economic traits of crops, such as grain yield, are complex and affected by a large number of genes, each with small effect, and thus the application of MAS in breeding is often less successful than expected. GS overcomes the challenges imposed by MAS, and has been proposed as a promising strategy in plant breeding for quantitative traits. Use of GEBVs rather than actual phenotypic values provides breeders the opportunity to select individual plants or animals for trait performance without doing actual phenotyping, thus potentially saving costs and time. This can be applied both to single, complex traits but also to multiple traits combined in an index. The possibility to estimate traits in an earlier stage is particularly advantageous in crops and animals with a long breeding cycle (e.g., tree breeding and cattle breeding), and, in this way, multiple years easily can be accelerated.

One major application of GS or any other methods that capture whole genotype/phenotype relationships in the breeding practice is the selection of parents for the next breeding cycle. This is done by prediction of a trait or an index of traits for all members of a panel of candidate parents (e.g., the GEBVs), after which the parents with the highest values are selected for further breeding, a practice not unlike the traditional selection practice based on actual phenotypes (Haley and Visscher, 1998). For further details of GS methods and techniques, see, e.g., Jannink, et al. Briefings in functional genomics, 2010: 9(2), 166-177, Goddard, et al. Journal of Animal breeding and Genetics 2007: 124 (6), 323-330, and Desta and Ortiz. Trends in plant science 2014:19(9), 592-601.

Conventionally, GS uses a set of individuals that is both phenotyped and genotyped (“the training set”) to train a statistical model that is applied to predict unobserved individuals (“the prediction set”) on the basis of having only genotyping data from the latter. The accuracy of GS to estimate GEBVs may be affected by multiple factors, one of them being the interaction of the genotypes (lines, or cultivars) with the environment (GxE), in both the training set and the predictions set.

The GxE effect in GS may be accounted for in statistical models. GS models incorporating GxE have been used in various crops such as wheat, corn, and legumes (see e.g., Burgueno et al, 2012; Cuevas et al, 2016; Cuevas et al, 2017; Jarquin et al, 2014; Jarquin et al, 2016; Jarquin et al, 2017; Roorkiwal et al, 2018; Saint Pierre at al, 2016; and Sukumaran et al, 2017). However, these GS models do not always account for the interaction between genetic markers and the environment, and when they do, the definition of environment is narrow, e.g., it is generally restricted to the factors of year and location. GS models incorporating “marker x environment” (MxE) interaction were proposed by Lopez Cruz et al in 2015 in wheat, which were later adopted by Crossa et al in 2016. Lopez Cruz et al (2015) evaluated wheat lines in environments resulting from a combination <af irrigation treatments, planting systems, planting date, and soil management practices over three years. Crossa et al (2016) referred to the environments as a combination of two growing seasons and three locations. In these models, GxE decomposes marker effects into components that are common across environments and specific to certain environment, enabling identification of genomic regions affecting E and GxE, respectively. In 2017, Cuevas et al introduced a modification to the “marker x environment” (MxE) model, but the authors still referred to the environments as a mere combination of years and locations.

Monteverde et al (2019) incorporated environment covariates into partial least square (PLS) and reaction norm models to predict plant traits in two rice breeding populations. However, those environment covariates only described weather properties (e.g., no soil or management practices information was incorporated), and were not subject to a clustering methodology to define envirotypes. In addition, the environment covariates used by Monteverde et al were not specified a priori on the parameter space of the statistical model.

Guillberg et al (2019) used soil and historical weather attributes in a GS model for barley varieties. However, such environmental information was directly incorporated into the GxE term of the statistical model, without defining envirotypes a priori.

More recently, He et al (2019) introduced environment covariates to a haplotype-based GS model for wheat lines. However, only weather-related attributes were considered when referring to an environment. In addition, He et al used a haplotype-based genomic relationship matrix, as opposed to e.g., a SNP-based matrix.

In comparison, the present invention differs from the aforementioned references in at least the following aspects: 1) the present invention takes into account of a broad range of environment information, such as weather attributes (e.g. temperature, precipitation, and solar radiation) that are grouped into four phenological stages from crop emergence to crop maturity, soil properties (e.g. texture, organic matter content, pH, bulk density, and available water capacity), and cropland information; 2) the present invention clusters the weather, soil, and cropland information a priori using k-means methodology by defining k number of envirotypes; 3) the present invention assigns year x location combinations from the training set to the corresponding pre-defined envirotype; 4) the present invention calculates marker effects specific to each envirotype to account for MxE; and 5) the present invention generates envirotype-specific genomic estimated breeding values (GEBVs).

The present invention is based, in part, on the surprising results that incorporation of envirotype information into genomic selection modeling can significantly increase accuracy and efficiency of genomic selection. Without wishing to be bound by any theory, the increased accuracy and efficiency of the present invention are, at least in part, the results of a better capture of the environmental effect on crop performance, particularly attributed by the following aspects of the present invention: 1) year x location combinations being assigned to envirotypes, which increases the number of data points per environment in the training set than what individual year x location combinations could have produced; 2) estimates of marker effects being specific to each envirotype, as opposed to being fixed and independent of the variation in the envirotypes; and 3) a wide range of environmental information being incorporated into envirotypes, such as weather attributes, soil properties, phenology, and cropland information.

Notably, the environment term in the GS model of the present invention may be determined a priori. For instance, the environment term in the GS model of the present invention may include G + E and G + E + GxE (or MxE) terms resulting from envirotypes built using weather, soil, and crop-related variables, clustered with a K-means methodology. In addition, envirotypes in the GS model of the present invention may utilize geo-referenced information, such that envirotype-specific GEBVs can be visualized on a map. Further, the statistical model of the present invention may utilize Bayesian statistics that are based on Bayes Theorem, as opposed to e.g., frequentist/classical statistics.

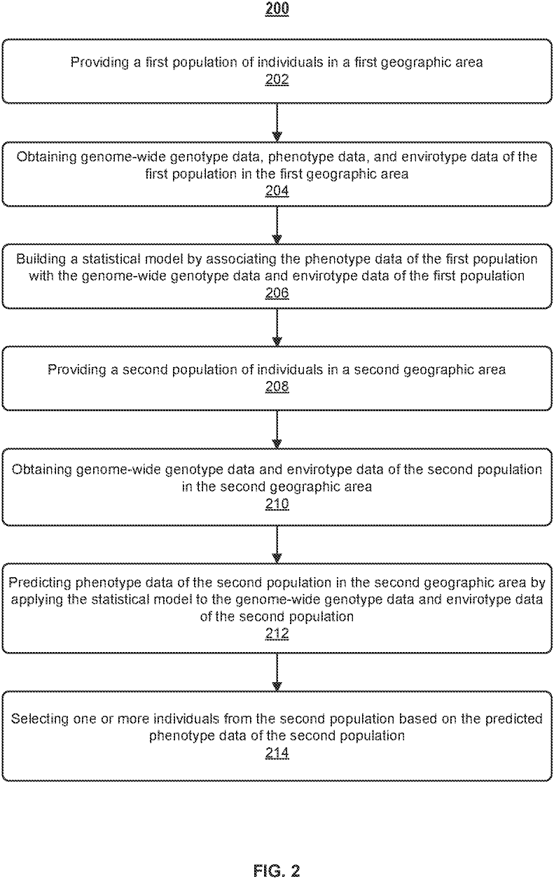

Accordingly, in certain aspect, provided herein is a method of genomic selection, including: providing a first population of individuals in a first geographic area; obtaining genome-wide genotype data, phenotype data, and envirotype data of the firsₜ population in the first geographic area; building a statistical model by associating the phenotype data of the first population with the genome-wide genotype data and envirotype data of the first population; providing a second population of individuals in a second geographic area; obtaining genome-wide genotype data and envirotype data of the second population in the second geographic area; predicting phenotype data of the second population in the second geographic area by applying the statistical model to the genome-wide genotype data and envirotype data of the second population: and selecting one or more individuals from the second population based on the predicted phenotype data of the second population, as illustrated in FIG. 2.

As used herein, the term “first population” refers to a population of individuals for the purposes of training or building a statistical model. The first population may include various suitable genetic materials. Examples of the genetic materials contained in the firsₜ population include, but are not limited to, inbred lines, segregating lines from a breeding population, and hybrids. In some embodiments, the first population is a genetically uniform population, such as a uniform cultivar population. In some embodiments, the first population is a genetically diverse population, comprising individuals with different genetic makeups.

As used herein, the term “second population” refers to a population of individuals for the purposes of predicting phenotype data. The second population may include various suitable genetic materials. Examples of the genetic materials contained in the second population include, but are not limited to, inbred lines, segregating lines from a breeding population, and hybrids. In some embodiments, the second population is a genetically diverse population. In some embodiments, the second population is a genetically uniform population. In some particular embodiments, the second population is an individual.

Various suitable individuals may be used in the present invention. In some embodiments, the individuals in the first population are inbred lines, breeding populations, or hybrids, and the individuals in the second population are segregating lines from breeding populations. In some embodiments, the individuals in the first population are hybrids, and the individuals in the second population are inbred lines and hybrids that may or may not have parental inbred lines in common with the hybrids from the first population.

With reference to FIG. 2, the selection step 214 may be of various suitable purposes. In some embodiments, the selection is for advancing the selected one or more individuals to a further stage in a breeding program. In some embodiments, the selection is for testing performance of the selected one or more individuals in a field. In some embodiments, the selected one or more individuals are segregating lines, inbred lines, or hybrid lines. In some embodiments, the selection is applied using a selection intensity.

In some embodiments, the method further includes producing offspring from the selected one or more individuals. With reference to FIG. 2, production of offspring may be added after the selection step of 214. In some embodiments, the offspring are produced by selfing, crossing, or asexual propagation. In some embodiments, the method further includes growing the offspring into maturity.

With reference to FIG. 2, the first population in 202 and the second population in 208 may be any suitable populations. In some embodiments, the first population is a training population and the second population is a prediction population or a target population. In some embodiments, the first population is a genetically uniform population. In some embodiments, the second population is a genetically diverse population. In some embodiments, the second population is a genetically uniform population. In some embodiments, the second population is an individual.

With reference to FIG. 2, the first geographic area in 202 and the second geographic area in 208 may be any suitable geographic areas. In some embodiments, the first geographic area and the second geographic area are the same geographic area. In some embodiments, the first geographic area and the second geographic area are different geographic areas. In some embodiments, the second geographic area is a target geographic area. In some embodiments, the target geographic area is a target breeding zone. In some embodiments, the target geographic area is a target market zone.

In some embodiments, the prediction quality of the built statistical model is tested on a third population from which both genotypes and phenotypes have been measured. The predictive ability of the model is determined by the correlation between the predicted estimate (e.g., GEBV) and the observed phenotypic value of the trait in a validation dataset. High correlation values indicate high prediction accuracy. Prediction accuracy depends on the heritability of the phenotype, as well as properties of both the training dataset and the validation dataset. With reference to FIG. 2, this step of testing prediction accuracy may be carried out between steps 206 and 208.

As used herein, building of a statistical mode! may include the initial establishment of the statistical model, training the statistical model, tuning the statistical mode!, validating the statistical model, and/or updating the statistical model. Various suitable statistical models may be used in the present invention. In some embodiments, the statistical model is a linear regression model, a logistic regression model, a Bayesian ridge regression model, a lasso regression model, an elastic net regression model, a decision tree model, a gradient boosted tree model, a neural network model, or a support vector machine model. Any suitable genomic selection algorithm may be used as the statistical model in the present invention. For further details of genomic selection algorithms and statistical models, see, e.g., Varshney, et al. Trends in biotechnology, 2009: 27(9), 522-530, Cardoso et al. Front Bioeng Biotechnol. 2015: 3:13, Ho et al. Frontiers in Genetics, 2019:10, and Azodi et al. G3: Genes, Genomes, Genetics 9.11 (2019): 3691-3702.

Accordingly, in certain aspect, the present invention provides a statistical model that is useful for genomic prediction and genomic selection. In some embodiments, the statistical model of the present invention comprises a genotype term, a phenotype term, and an environment term. In some embodiments, the statistical model further comprises a genotype by environment (GxE) term. In some embodiments, the genotype term in the statistical model comprises a SNP-based genomic relationship matrix. In some embodiments, the environment term comprises one or more envirotypes, wherein the one or more envirotypes comprise data on time, location, weather, soil, companion organism, management, crop canopy, cultivation area, or a combination thereof. In some embodiments, the statistical model of the present invention is a Bayesian model. In some embodiments, the one or more envirotypes of the present invention are determined a priori in the statistical model. In some embodiments, the one or more envirotypes are clustered by a clustering methodology. In some embodiments, the clustering methodology is a K-means clustering methodology.

### Envirotype

Envirotype refers to the characterization of the environmental factors that affect the phenotypic expression of traits, complementing genotype and phenotype. Envirotyping refers to the process of obtaining and characterizing the environment factors (e.g., year, location, and management) that are experienced in a geography. Envirotype information may be useful for: definition of breeding zones: definition of product market zones; understanding GxE interaction: identification of trial locations for multi-environmental trials (METs) that would serve to generate training sets for genomic predictions: and identification of targeted population of environments (TPE) for future trialing aimed at training set creation, aligned with breeding and market zones’ envirotype. Further reference of envirotype and envirotyping methods and techniques may be made to, e.g., Xu, Yunbi. Theoretical and Applied Genetics 129.4 (2016): 653-673.

Accordingly, the envirotype data of the present invention may contain information from various environmental factors that could have an effect on the growth and/or development of a plant or an animal. In some embodiments, the envirotype data is time data, location data, weather data, soil data, companion organism data, management data, crop canopy data, cultivation area data, or a combination thereof.

Various suitable time, location, and geographic data may be used for the present invention. In some embodiments, the time data is century, decade, year, season, month, day, hour, minute, second, or a combination thereof. For instance, the envirotype may be a monthly average of precipitation in the breeding zone. In some embodiments, the location data is latitude, longitude, altitude, or a combination thereof. For instance, geographic information system (GIS) data may be used as envirotype data. GIS has been established with the merging of cartography, statistical analysis, and database technology, which is designed for collecting, storing, integrating, analyzing, and managing all types of geographical data. The data for any location in Earth space-time can be collected as dates/times of occurrence, with longitude, latitude, and elevation determined by x, y, and z coordinates, respectively. GIS integrates various data sources with existing maps and up-to-date records from climate satellites. To capture climate data, various types of weather observatory stations have been established worldwide, including ground, radiosonde, wind, rocket, radiation, agrometeorological, and automatic weather stations. These stations document climate data for numerous locations and sites, which are transferred in international or national central databases and become a part of GIS data.

Various suitable weather data may be used for the present invention. In some embodiments, the weather data is temperature, humidity, pressure, zonal wind speed, meridional wind speed, long-wave radiation, fraction of total precipitation that is convective, convective available potential energy, potential evaporation, precipitation hourly total, short-wave solar radiation, photoperiod, or a combination thereof. Weather data can be obtained from NASA (NLDAS primary forcing data). See David Mocko, NASA/GSFC/HSL (2012) NLDAS Primary Forcing Data L4 Monthly 0.125 × 0.125 degree V002, Greenbelt, Maryland, USA, Goddard Earth Sciences Data and Information Services Center (GES DISC), and Xia et al. (2012) Continental-scale water and energy flux analysis and validation for the North American Land Data Assimilation System project phase 2 (NLDAS-2): 1. Intercomparison and application of model products, J. Geophys. Res., 117, D03109. In some embodiments, the envirotype data may include photoperiod information, which would be relevant for crops or varieties that are photoperiod sensitive.

Various suitable soil data may be used for the present invention. In some embodiments, the soil data is soil type, soil structure, soil moisture, soil depth, soil organic matter content, soil density, soil pH, soil fertility, soil salinity, or a combination thereof. Soil is generally characterized by its texture, defined by the percentage of clay, silt, and sand. Data may be broken down by soil depth and/or map units. It can be useful to aggregate data, to obtain weighted soil composition data for each grid unit. Other soil attributes that are used include organic matter, pH, bulk density, and available water capacity. Soil data can be obtained from any suitable source, such as the SSURGO database from the United States Department of Agriculture (USDA).

Various suitable companion organism data may be used for the present invention. In some embodiments, the companion organism data is soil fauna, insects, animals, weeds, or a combination thereof. Companion organisms are those surrounding crop plants, including bacteria, fungi, viruses, insects, weeds, and even other intercropping plants, which should be considered an important component of the environments. A series of methods and protocols have been developed to measure or determine companion organisms for different crops through multidisciplinary collaborations. For example, rhizospheric microorganisms can be extracted from bulked soil samples followed by comprehensive analysis and evaluation. Bulked sample analysis combined with metagenomics and DNA- or RNA-seq can be used to determine precisely the species, quantity, and mutual relationships of the organisms in bulked soil samples (Myrold et al. 2014). Using bulked samples collected from leaves or crop canopy, the organisms on the plant surface can be analyzed for their species, quantity, origin, distribution, developmental stages, and possible symbiontic relationships.

Various suitable management data may be used for the present invention. Crop management, as a unique environment component, involves intercropping, rotating, and agronomic practices. Environmental factors that affect plant growth and yield can be modified or dramatically changed by human management activities. In some embodiments, the management data is intercropping management, cover-cropping management, rotating cropping management, or a combination thereof.

Further, a variety of suitable crop canopy data may be used for the present invention. In some embodiments, the crop canopy data is obtained from an aerial platform. Remote sensing techniques, such as spectroradiometrical reflectance, digital imagery, thermal images, near Infrared reflectance spectroscopy, and infrared photography, provide tools for characterization of crop canopy. These tools can be used with an airborne remote sensing platform to collect data for temperature, humidity, light, air, biomass, and overage of the crop canopy. Robotic imaging platforms and computer vision-assisted analytical tools developed for high-throughput plant phenotyping (Fahlgren et al. 2015) can be used for measurement of the crop canopy. Automated recovery of three-dimensional models of plant shoots can be used for multiple color images (Pound et al. 2014). The 3-D structure can be also determined directly using laser scanning (Paulus et al. 2013) and deep time-flight sensor (Chene et al. 2012).

In some embodiments, the envirotype data is grouped according to the growth stages of the individuals. In some embodiments, only those months when a particular crop grows and developed are used to build envirotypes. For example, in constructing an envirotype model for maize, it can be useful to group weather attributes in four stages from planting to physiological maturity: 1) planting-V7, 2) V7-R1, 3) R1-R3, and 4) R3-R6, wherein the Vs refer to the vegetative stages and Rs refer to the reproductive stages. Methods and techniques for assessing plant growth and development stages are known in the art. For instance, reference of corn (maize) growth stages may be made to McWilliams, Denise A., Duane Raymond Berglund, and G. J. Endres. “Corn growth and management quick guide.” (1999).

It is contemplated that the envirotype data of the present invention may be collected, combined, and compiled into an envirotype map. In some embodiments, the envirotype data is an envirotype map. A useful envirotype map can be built by associating similar areas of a geographic map, such as the 48 contiguous U.S. states or the more restricted soybean and corn growing regions, with relevant environmental conditions underlying the respective regions. Accordingly, a grid can be constructed based on the resolution of the environmental data employed to build the envirotype map. For example, each pixel or basic grid area of the map can be an area of about 14 square kilometers. An envirotype map can be built using any one of the above-mentioned environmental factors (e.g., weather and soil attributes), or a combination thereof.

Cultivation area information can be obtained from USDA National Agricultural Statistics Service database. Accordingly, in some embodiments, to determine the limits of the envirotype map, a cropland data layer can be made by filtering out areas irrelevant to production of a crop of interest, such as corn or soy.

To facilitate statistical analysis, in some embodiments, the envirotype is clustered. The weather data, soil data, or weather and soil grids can be clustered using different methodologies, such as Kmeans. Resulting clusters define envirotypes. The envirotypes can then be used as covariate in the genetic model to predict crop performance based on the genetic profile of each cultivar. By way of example, a GxE (“genotype by environment”) Bayesian ridge regression model can be built using collected phenotypic data, for example, grain yield, as well as genome-wide genetic data (molecular DNA information).

### Variety Development and Breeding

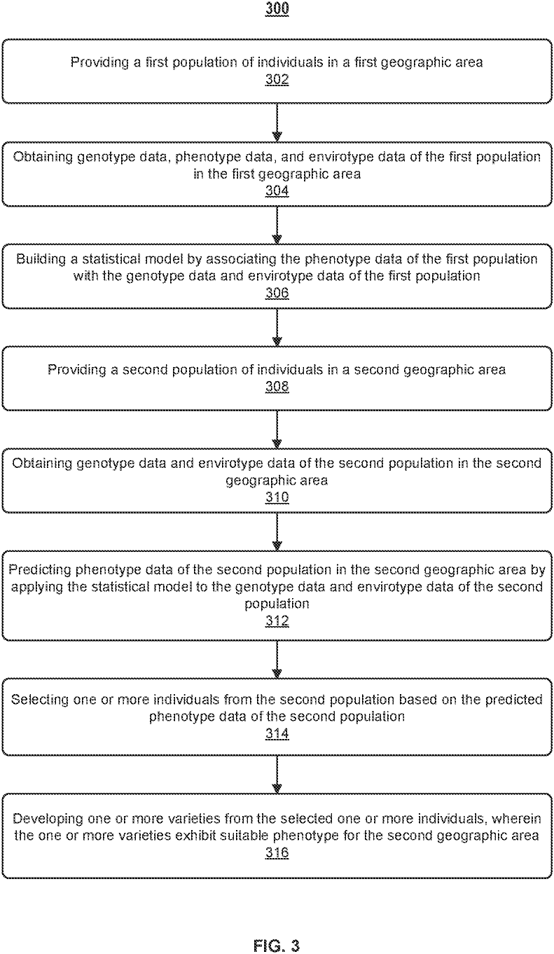

The present invention may be used for variety development. Accordingly, in yet another aspect, provided herein is a method for developing one or more varieties suitable for a geographic area, including: providing a first population of individuals in a first geographic area; obtaining genotype data, phenotype data, and envirotype data of the first population in the first geographic area; building a statistical model by associating the phenotype data of the first population with the genotype data and envirotype data of the first population; providing a second population of individuals in a second geographic area; obtaining genotype data and envirotype data of the second population in the second geographic area: predicting phenotype data of the second population in the second geographic area by applying the statistical model to the genotype data and envirotype data of the second population; selecting one or more individuals from the second population based on the predicted phenotype data of the second population; and developing one or more varieties from the selected one or more individuals, wherein the one or more varieties exhibit suitable phenotype for the second geographic area, as illustrated in FIG. 3.

Various methods and techniques of variety development in plants and animals are known in the art and may be used in the present invention. By way of example, in plant variety development, the development of a commercial hybrid plant variety involves the development of parental inbred varieties, the crossing of these parental inbred varieties, and the evaluation of the hybrid crosses. A plant breeder can initially select and cross two or more parental lines to produce hybrid lines from which to select. This can be followed by repeated selfing and selection, in order to produce many new genetic combinations. Moreover, a breeder can generate multiple different genetic combinations by crossing, selfing, and mutations. A plant breeder can select which germplasm to advance to the next generation. This germplasm may then be grown under different geographical, climatic, and soil conditions, and further selections can be made.

With reference to FIG. 3, in some embodiments, the individuals in the first population in 302 are inbred lines, and the individuals in the second population in 308 are hybrid lines. In some embodiments, the individuals in the first population in 302 are parental lines and the individuals in the second population in 308 are filial lines derived from the parental lines.

With reference to FIG. 3, in some embodiments, the selection in 314 is for advancing the selected one or more individuals to a further stage in a breeding program. In some embodiments, the selection in 314 is for testing performance of the selected one or more individuals in a field. In some embodiments, the selected one or more individuals in 314 are segregating lines, inbred lines, or hybrid lines. In some embodiments, the selection is applied using a selection intensity.

With reference to FIG. 3, in some embodiments, the method further includes producing offspring from the one or more developed varieties in 316. In some embodiments, the offspring are produced by selfing, crossing, or asexual propagation. In some embodiments, the method further includes growing the offspring into maturity.

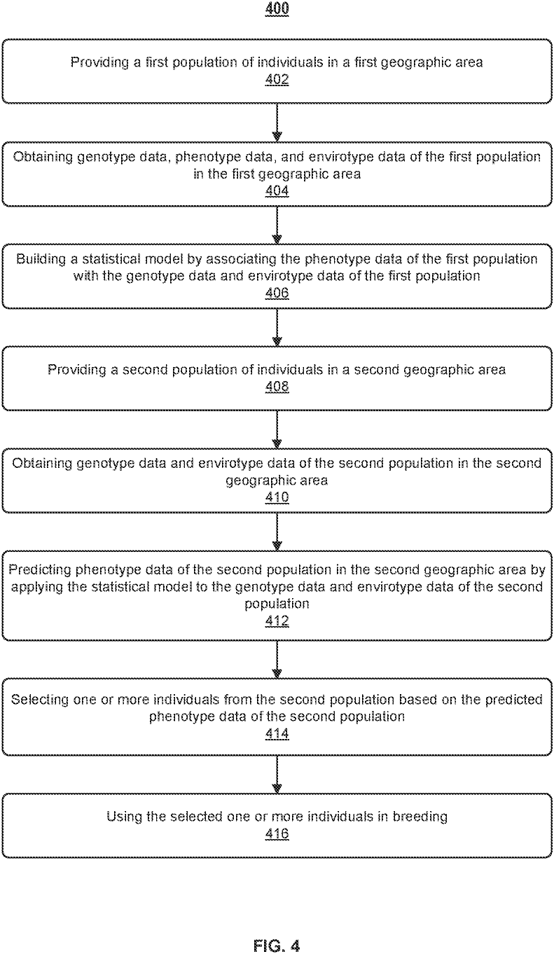

Moreover, the present invention may be used for various types of breeding. Accordingly, in still another aspect, provided herein is a method of breeding, including: providing a first population of individuals in a first geographic area; obtaining genotype data, phenotype data, and envirotype data of the first population in the first geographic area; building a statistical model by associating the phenotype data of the first population with the genotype data and envirotype data of the first population: providing a second population of individuals in a second geographic area; obtaining genotype data and envirotype data of the second population in the second geographic area; predicting phenotype data of the second population in the second geographic area by applying the statistical model to the genotype data and envirotype data of the second population: selecting one or more individuals from the second population based on the predicted phenotype data of the second population; and using the selected one or more individuals in breeding, as illustrated in FIG. 4.

Various methods and techniques of plant and animal breeding are known in the art and may be used in the present invention. With reference to FIG. 4, this breeding step may be carried out in step 416.

For instance, pedigree breeding is commonly used for the improvement of self-pollinating crops or inbred lines of cross-pollinating crops. Two parents (e.g., two individuals selected from the step 414 in FIG. 4) that possess favorable, complementary traits are crossed to produce an F₁. An F₂ population is produced by selfing one or several F₁’s or by intercrossing two F₁’s (sib mating). Selection of the best individuals is usually begun in the F₂ population. Then, beginning in the F₃, the best individuals in the best families are selected. Replicated testing of families, or hybrid combinations involving individuals of these families, often follows in the F₄ generation to improve the effectiveness of selection for traits with low heritability. At an advanced stage of inbreeding (i.e., F₆ and F₇), the best lines or mixtures of phenotypically similar lines are tested for potential release as new varieties.

Mass and recurrent selections can be used to improve populations of either self- or cross-pollinating crops. A genetically variable population of heterozygous individuals is either identified or created by intercrossing several different parents. The best plants are selected based on individual superiority, outstanding progeny, or excellent combining ability. The selected plants are intercrossed to produce a new population in which further cycles of selection are continued.

Backcross breeding may be used to transfer genes for a simply inherited, highly heritable trait into a desirable homozygous cultivar or line that is the recurrent parent. The source of the trait to be transferred is called the donor parent. The resulting plant is expected to have the attributes of the recurrent parent and the desirable trait transferred from the donor parent. After the initial cross, individuals possessing the phenotype of the donor parent are selected and repeatedly crossed (backcrossed) to the recurrent parent. The resulting plant is expected to have the attributes of the recurrent parent and the desirable trait transferred from the donor parent.

The single-seed descent procedure in the strict sense refers to planting a segregating population, harvesting a sample of one seed per plant, and using the one-seed sample to plant the next generation. When the population has been advanced from the F₂ to the desired level of inbreeding, the plants from which lines are derived will each trace to different F₂ individuals. The number of plants in a population declines with each generation due to failure of some seeds to germinate or some plants to produce at least one seed. As a result, not all of the F₂ plants originally sampled in the population will be represented by a progeny when generation advance is completed.

Molecular markers can also be used during the breeding process for the selection of qualitative traits. For example, markers closely linked to alleles or markers containing sequences within the actual alleles of interest can be used to select plants that contain the alleles of interest during a backcrossing breeding program. The markers can also be used to select toward the genome of the recurrent parent and against the markers of the donor parent. This procedure attempts to minimize the amount of genome from the donor parent that remains in the selected plants. It can also be used to reduce the number of crosses back to the recurrent parent needed in a backcrossing program. The use of molecular markers in the selection process is often called genetic marker-enhanced selection or MAS. Molecular markers may also be used to identify and exclude certain sources of germplasm as parental varieties or ancestors of a plant by providing a means of tracking genetic profiles through crosses.

Mutation breeding may also be used to introduce new traits into a variety. Mutations that occur spontaneously or are artificially induced can be useful sources of variability for a plant breeder. The goal of artificial mutagenesis is to increase the rate of mutation for a desired characteristic. Mutation rates can be increased by many different means including temperature, long-term seed storage, tissue culture conditions, radiation (such as X-rays, Gamma rays, neutrons, Beta radiation, or ultraviolet radiation), chemical mutagens (such as base analogs like 5-bromo-uracil), antibiotics, alkylating agents (such as sulfur mustards, nitrogen mustards, epoxides, ethyleneamines, sulfates, sulfonates, sulfones, or lactones), azide, hydroxylamine, nitrous acid, or acridines. Once a desired trait is observed through mutagenesis, the trait may then be incorporated into existing germplasm by traditional breeding techniques. Details of mutation breeding can be found in Principles of Cultivar Development by Fehr, Macmillan Publishing Company (1993).

The production of double haploids can also be used for the development of homozygous varieties in a breeding program. Double haploids are produced by the doubling of a set of chromosomes from a heterozygous plant to produce a completely homozygous individual. For example, see Wan, et al., Theor. Appl. Genet., 77:889-892 (1989).

Genetic engineering tools such as transgenic and genome-editing techniques may also be used for variety development and breeding. See, e.g., Moose, Stephen P., and Rita H. Mumm. “Molecular plant breeding as the foundation for 21st century crop improvement.” Plant physiology 147.3 (2008): 969-977, and Chen, Kunling, et al. “CRISPR/Cas genome editing and precision plant breeding in agriculture.” Annual review of plant biology 70 (2019): 667-697.

Additional non-limiting examples of plant variety development and breeding methods that may be used include, without limitation, those found in Principles of Plant Breeding, John Wiley and Son, pp. 115-161 (1960): Allard (1960); Simmonds (1979); Sneep, et al. (1979): Fehr (1987); and “Carrots and Related Vegetable Umbelliferae”, Rubatzky, V.E., et al. (1999).

For further details of methods and techniques in animal variety development and breeding, see, e.g., Misztal 1. (2013) Animal Breeding and Genetics, Introduction. In: Christou P., Savin R., Costa-Pierce B.A., Misztal I., Whitelaw C.B.A. (eds) Sustainable Food Production. Springer, New York, NY.

It is contemplated that the method of variety development or breeding as described herein may be used in any suitable species. In some embodiments, the one or more individuals are a crop selected from the group consisting of maize, soybean, wheat, sorghum, barley, oats, rice, millet, canola, cotton, cassava, cowpea, safflower, sesame, tobacco, flax, sunflower, a grain crop, a vegetable crop, an oil crop, a forage crop, an industrial crop, a woody crop, and a biomass crop.

In some embodiments, the one or more individuals are selected from the group consisting of cattle, sheep, pigs, goats, horses, mice, rats, rabbits, cats, and dogs.

In certain aspects, the present invention provides a variety developed by any one of the methods disclosed herein. In some particular embodiments, the developed variety is a hybrid corn variety.

### Systems for Genomic Prediction and Selection Using Envirotype Data

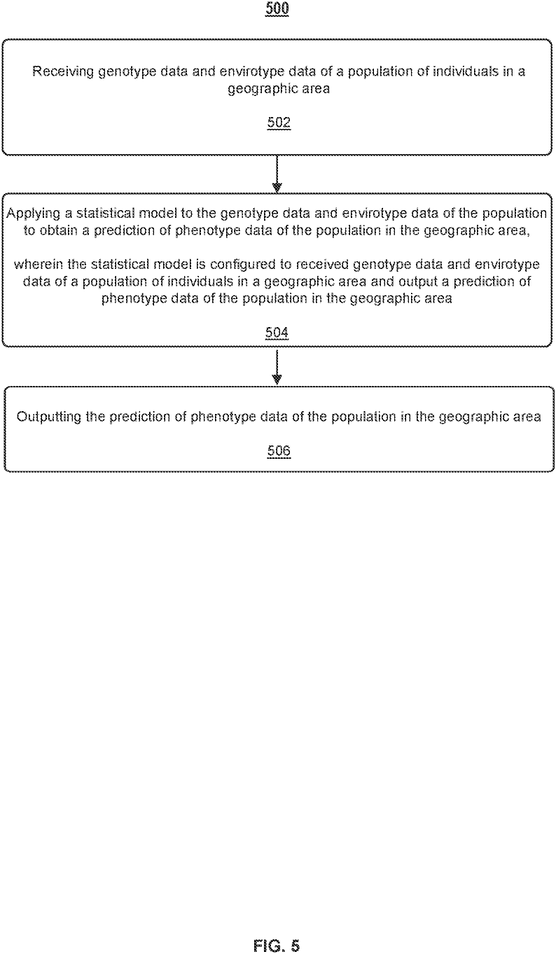

In still another aspect, provided herein is a computer-implemented method for predicting phenotype data of a population in a geographic area, including: receiving genotype data and envirotype data of a population of individuals in a geographic area; and applying a statistical model to the genotype data and envirotype data of the population to obtain a prediction of phenotype data of the population in the geographic area, wherein the statistical model is configured to receive genotype data and envirotype data of a population of individuals in a geographic area and output a prediction of phenotype data of the population in the geographic area; and outputting the prediction of phenotype data of the population in the geographic area, as illustrated in FIG. 5.

With reference to FIG. 5, in some embodiments, after step 506, the method further includes selecting one or more individuals from the population based on the predicted phenotype data of the population. In some embodiments, the method further comprises informing a user of the selected one or more individuals for breeding.

In some embodiments, the statistical model is a trained model. For instance, the model has been previous trained with a training population. Various suitable statistical models may be used in the present invention. Relevant statistical models and algorithms include, but are not limited to, discriminant analysis including linear, logistic, and more flexible discrimination techniques (see, e.g., Gnanadesikan, 1977, Methods for Statistical Data Analysis of Multivariate Observations, New York: Wiley 1977); tree-based algorithms such as classification and regression trees (CART) and variants (see, e.g., Breiman, 1984, Classification and Regression Trees, Belmont, Calif.: Wadsworth International Group); generalized additive models (see, e.g., Tibshirani, 1990, Generalized Additive Models, London: Chapman and Hall): and neural networks (see, e.g., Neal, 1996, Bayesian Learning for Neural Networks, New York: Springer-Verlag; and Insua, 1998, Feedforward neural networks for nonparametric regression In: Practical Nonparametric and Semi parametric Bayesian Statistics, pp. 181-194, New York: Springer). Further examples of on the various genomic selection algorithms may be referred to, for instance, Azodi, Christina B., et al. “Benchmarking algorithms for genomic prediction of complex traits.” bioRxiv (2019): 614479. Accordingly, in some embodiments, the statistical model in step 504 is a linear regression model, a logistic regression model, a Bayesian ridge regression model, a lasso regression model, an elastic net regression model, a decision tree model, a gradient boosted tree model, a neural network model, or a support vector machine model.

Any of the aforementioned methods of present invention may be implemented as computer program processes that are specified as a set of instructions recorded on a computer-readable storage medium (also referred to as a computer-readable medium-CRM).

Accordingly, in yet still another aspect, provided herein is a non-transitory computer-readable storage medium storing one or more programs, the one or more programs comprising instructions, which when executed by one or more processors of an electronic device having a display, cause the electronic device to: receiving genotype data and envirotype data of a population of individuals in a geographic area; and applying a statistical model to the genotype data and envirotype data of the population to obtain a prediction of phenotype data of the population in the geographic area, wherein the statistical model is configured to receive genotype data and envirotype data of a population of individuals in a geographic area and output a prediction of phenotype data of the population in the geographic area; and outputting the prediction of phenotype data of the population in the geographic area.

Examples of computer-readable storage media include RAM, ROM, read-only compact discs (CD-ROM), recordable compact discs (CD-R), rewritable compact discs (CD-RW), read-only digital versatile discs (e.g., DVD-ROM, dual-layer DVD-ROM), a variety of recordable/rewritable DVDs (e.g., DVD-RAM, DVD-RW, DVD+RW, etc.), flash memory (e.g., SD cards, mini-SD cards, micro-SD cards, etc.), magnetic and/or solid state hard drives, ultra-density optical discs, any other optical or magnetic media, and floppy disks. In some embodiments, the computer-readable storage medium is a solid-state device, a hard disk, a CD-ROM, or any other non-volatile computer-readable storage medium.

The computer-readable storage media can store a set of computer-executable instructions (e.g., a “computer program”) that is executable by at least one processing unit and includes sets of instructions for performing various operations.

A computer program (also known as a program, software, software application, script, or code) can be written in any form of programming language, including compiled or interpreted languages, declarative or procedural languages, and it can be deployed in any form, including as a standalone program or as a module, component, or subroutine, object, or other component suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, subprograms or portions of code). A computer program can be deployed to be executed on one computer or on multiple computers that are located at one site or distributed across multiple sites and interconnected by a communication network. Examples of computer programs or computer code include machine code, such as is produced by a compiler, and files including higher-level code that are executed by a computer, an electronic component, or a microprocessor using an interpreter.

As used herein, the term “software” is meant to include firmware residing in read-only memory or applications stored in magnetic storage, which can be read into memory for processing by a processor. Also, in some implementations, multiple software aspects of the subject disclosure can be implemented as sub-parts of a larger program while remaining distinct software aspects of the subject disclosure. In some implementations, multiple software aspects can also be implemented as separate programs. Any combination of separate programs that together implement a software aspect described here is within the scope of the subject disclosure. In some implementations, the software programs, when installed to operate on one or more electronic systems, define one or more specific machine implementations that execute and perform the operations of the software programs.

Further, any one of the preceding methods of the present invention may be implemented in one or more computer systems or other forms of apparatus. Examples of apparatus include but are not limited to, a computer, a tablet personal computer, a personal digital assistant, and a cellular telephone. Accordingly, provided herein is an electronic device, comprising: a display: one or more processors; a memory; and one or more programs, wherein the one or more programs are stored in the memory and configured to be executed by the one or more processors, the one or more programs including instructions for: receiving genotype data and envirotype data of a population of individuals in a geographic area; and applying a statistical model to the genotype data and envirotype data of the population to obtain a prediction of phenotype data of the population in the geographic area, wherein the statistical model is configured to receive genotype data and envirotype data of a population of individuals in a geographic area and output a prediction of phenotype data of the population in the geographic area; and outputting the prediction of phenotype data of the population in the geographic area.

In some embodiments, the electronic device may be a server computer, a client computer, a personal computer (PC), a user device, a tablet PC, a laptop computer, a personal digital assistant (PDA), a cellular telephone, or any machine capable of executing a set of instructions, sequential or otherwise, that specify actions to be taken by that machine. In some embodiments, the electronic device may further include keyboard and pointing devices, touch devices, display devices, and network devices.

As used herein, the terms “computer”, “processor”, and “memory” all refer to electronic or other technological devices. These terms exclude people or groups of people. For the purposes of the specification, the terms “display” or “displaying” means displaying on an electronic device. As used in this specification and any claims of this application, the terms “computer readable medium” and “computer readable media” are entirely restricted to tangible, physical objects that store information in a form that is readable by a computer. These terms exclude any wireless signals, wired download signals, and any other ephemeral signals.

To provide for interaction with a user, implementations of the subject matter described in this specification can be implemented on a computer having a display device described herein for displaying information to the user and a virtual or physical keyboard and a pointing device, such as a finger, pencil, mouse or a trackball, by which the user can provide input to the computer. Other kinds of devices can be used to provide for interaction with a user as well; for example, feedback provided to the user can be any form of sensory feedback, such as visual feedback, auditory feedback, or tactile feedback; and input from the user can be received in any form, including acoustic, speed, or tactile input.

FIG. 6 illustrates an example of the electronic device. Device 600 can be a host computer connected to a network. Device 600 can be a client computer or a server. As shown in FIG. 6, device 600 can be any suitable type of microprocessor-based device, such as a personal computer, workstation, server or handheld computing device (portable electronic device) such as a phone or tablet. The device can include, for example, one or more of processor 610, input device 620, output device 630, storage 640, and communication device 660. Input device 620 and output device 630 can generally correspond to those described above, and can either be connectable or integrated with the computer.

Input device 620 can be any suitable device that provides input, such as a touch screen, keyboard or keypad, mouse, or voice-recognition device. Output device 630 can be any suitable device that provides output, such as a touch screen, haptics device, or speaker.

Storage 640 can be any suitable device that provides storage, such as an electrical, magnetic or optical memory including a RAM, cache, hard drive, or removable storage disk. Communication device 660 can include any suitable device capable of transmitting and receiving signals over a network, such as a network interface chip or device. The components of the computer can be connected in any suitable manner, such as via a physical bus or wirelessly.

Software 650, which can be stored in storage 640 and executed by processor 610, can include, for example, the programming that embodies the functionality of the present disclosure (e.g., as embodied in the devices as described above).

Software 650 can also be stored and/or transported within any non-transitory computer-readable storage medium for use by or in connection with an instruction execution system, apparatus, or device, such as those described above, that can fetch instructions associated with the software from the instruction execution system, apparatus, or device and execute the instructions. In the context of this disclosure, a computer-readable storage medium can be any medium, such as storage 640, that can contain or store programming for use by or in connection with an instruction execution system, apparatus, or device.

Software 650 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate or transport programming for use by or in connection with an instruction execution system, apparatus, or device. The transport readable medium can include, but is not limited to, an electronic, magnetic, optical, electromagnetic or infrared wired or wireless propagation medium.

Device 600 may be connected to a network, which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Device 600 can implement any operating system suitable for operating on the network. Software 650 can be written in any suitable programming language, such as C, C++, Java or Python. In various embodiments, application software embodying the functionality of the present disclosure can be deployed in different configurations, such as in a client/server arrangement or through a Web browser as a Web-based application or Web service, for example.

Although the disclosure and examples have been fully described with reference to the accompanying figures, it is to be noted that various changes and modifications will become apparent to those skilled in the art. Such changes and modifications are to be understood as being included within the scope of the disclosure and examples as defined by the claims.

The foregoing description, for purpose of explanation, has been described with reference to specific embodiments. It is understood that any specific order or hierarchy of blocks in the processes disclosed is an illustration of example approaches. Based upon design preferences, it is understood that the specific order or hierarchy of blocks in the processes may be rearranged, or that all illustrated blocks be performed. Some of the blocks may be performed simultaneously. For example, in some instances, multitasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products. Others skilled in the art are thereby enabled to best utilize the techniques and various embodiments with various modifications as are suited to the particular use contemplated.

### EXAMPLES

The following examples are offered to illustrate provided embodiments and are not intended to limit the scope of the present disclosure.

### Example 1: Increased Effectiveness of Genomic Selection Based on Envirotype Model Predictions

This example illustrates a crop product development project aiming at making a new high-yielding corn (Zea mays) hybrid variety that is better suited for cultivation at a specific location.

Genotype data for a population of available candidate parental inbred lines were collected, but not all potential hybrid combinations were phenotypically observed and tested in the field at the specific location. Thus, this population of all candidate parental inbred lines and all potential hybrid combinations was the prediction population.

Three genomic selection models were built: Model 1, which only utilized genotype information in the form of G term; Model 2, which included genotype and envirotype information in the form of G + E terms and assumed all genetic markers in the G term having the same effect across all the envirotypes in the E term (i.e. a common genomic relationship matrix is applied across all envirotypes); and Model 3, which included genotype, envirotype, and genotype x envirotype interaction information in the form of G + E + GxE terms and assumed that the effect of the genetic markers in the G term varies across envirotypes in the E term (i.e. a genomic relationship matrix specific to each envirotype is built when estimating the effect of genotype x envirotype interaction).

Envirotypes were defined by using: i) 40 years of historical weather data (1978-2018), including information on average temperature, accumulated precipitation, and solar radiation, all computed on a monthly basis and grouped into four stages of corn growth and development from vegetative (V) to reproductive (R), including VE (vegetative emergence) to V7 (7^{th} leave present), V7 to R1 (silking stage), R1 to R3 (kernel milk stage), and R3 to R6 (physiological maturity stage), see corn growth and development stages in McWilliams et al., “Corn growth and management quick guide”, 1999; ii) soil attribute data, including texture (% sand, % silt, % clay), organic matter percentage, pH, bulk density, and available water capacity; and iii) cropland data from areas that were planted with greater than or equal to 5% of corn or soybean in the U.S. in 2017. These weather, soil, and cropland data were clustered using k-means method with k set to 4-20, and the specified k value determined the number of pre-defined envirotypes obtained.

These three models were trained with a common training population of hybrids, for which both genotype data and field performance (phenotype) data on the hybrids and their parental inbred lines were collected from various geographic testing locations in the U.S. in 2014 and 2015. The coordinates of the various geographic testing locations in each of the two years were used to assign them to the corresponding pre-defined envirotypes. This dataset was the training dataset.

The models were trained and applied to the common set of candidate parental inbred lines that had genotype data available. Genomic estimated breeding values (GEBVs) were calculated for all possible hybrid combinations from these parental inbred lines in the target specific location in 2016. After the 2016 field season, the hybrids were harvested and grain yield data were obtained.

Results showed that with Model 1, which only used genotype information with G term, the correlation between the prediction and the actual harvested grain yield data in 2016 was 0.20. In comparison, with Model 2, which included genotype and envirotype information in the form of G + E terms and assumed all genetic markers in the G term having the same effect across all the envirotypes in the E term, the correlation between the prediction and the actual harvested grain yield in 2016 was 0.30. With Model 3, which included genotype, envirotype, and genotype x envirotype interaction information in the form of G + E + GxE terms and assumed that the effect of the genetic markers in the G term varies across envirotypes in the E term, the correlation between the prediction and the actual harvested grain yield data in 2016 was 0.31 averaged across envirotypes. Thus, compared to Model 1, Model 2 and Model 3 represent a 50% and a 55% increase in prediction accuracy, respectively. A selection intensity was then applied to select, based on the predicted GEBV values, the top ranked hybrid combinations in each target location for future testing sets. The selection intensity used was conditional to the predictive ability of the model, as well as the field resources available for testing the top predicted hybrids.

It is known that the accuracy of genomic prediction is affected by a number of factors, including the heritability of the trait, as well as the method of modeling. For a low heritability trait like grain yield in corn, the accuracy of genomic selection is generally low (see, e.g. Jia and Jean-Luc. Genetics 192.4 (2012): 1513-1522, Zhao et al. Theoretical and Applied Genetics 124.4 (2012): 769-776, and Zhang et al. Frontiers in plant science 8 (2017): 1916). Results of this example show that by incorporating a wide variety of envirotype information into genomic selection modeling, the prediction accuracy can be greatly increased. Specifically, it is shown here that incorporation of weather, soil, and cropland envirotypes into genomic selection modeling surprisingly increased the prediction accuracy by 50%-55%.

Thus, this example demonstrates successful development of a new high-yielding corn hybrid variety that is better suited for cultivation at a specific location. Similarly, a project aiming at identifying the best segregating line among sister lines from a female or male breeding population, or a project aiming at coding the best finished inbred lines, can utilized a similar model to assist selections with GEBV specific to target breeding zones and/or market geographies.

1. A method of breeding, comprising:
a) providing a first population of individuals in a first geographic area;
b) obtaining genotype data, phenotype data, and envirotype data of the first population in the first geographic area;
c) building a statistical model by associating the phenotype data of the first population with the genotype data and envirotype data of the first population;
d) providing a second population of individuals in a second geographic area;
e) obtaining genotype data and envirotype data of the second population in the second geographic area;
f) predicting phenotype data of the second population in the second geographic area by applying the statistical model to the genotype data and envirotype data of the second population;
g) selecting one or more individuals from the second population based on the predicted phenotype data of the second population; and
h) using the selected one or more individuals in breeding.

2. A method for predicting phenotype data of a population in a geographic area for use in breeding, comprising:
a) providing a first population of individuals in a first geographic area;
b) obtaining genotype data, phenotype data, and envirotype data of the first population in the first geographic area;
c) building a statistical model by associating the phenotype data of the first population with the genotype data and envirotype data of the first population;
d) providing a second population of individuals in a second geographic area;
e) obtaining genotype data and envirotype data of the second population in the second geographic area; and
f) predicting phenotype data of the second population in the second geographic area by applying the statistical model to the genotype data and envirotype data of the second population.

3. The method of claim 2, further comprising selecting one or more individuals from the second population based on the predicted phenotype data of the second population;
and using the selected one or more individuals in breeding.

4. A method of genomic selection, comprising:
a) providing a first population of individuals in a first geographic area;
b) obtaining genome-wide genotype data, phenotype data, and envirotype data of the first population in the first geographic area;
c) building a statistical model by associating the phenotype data of the first population with the genome-wide genotype data and envirotype data of the first population;
d) providing a second population of individuals in a second geographic area;
e) obtaining genome--wide genotype data and envirotype data of the second population in the second geographic area;
f) predicting phenotype data of the second population in the second geographic area by applying the statistical model to the genome-wide genotype data and envirotype data of the second population; and
g) selecting one or more individuals from the second population based on the predicted phenotype data of the second population.

5. The method of claim 4, further comprising: using the selected one or more individuals in breeding.

6. A method for developing one or more varieties suitable for a geographic area, comprising:
a) providing a first population of individuals in a first geographic area;
b) obtaining genotype data, phenotype data, and envirotype data of the first population in the first geographic area;
c) building a statistical model by associating the phenotype data of the first population with the genotype data and envirotype data of the first population;
d) providing a second population of individuals in a second geographic area;
e) obtaining genotype data and envirotype data of the second population in the second geographic area;
f) predicting phenotype data of the second population in the second geographic area by applying the statistical model to the genotype data and envirotype data of the second population;
g) selecting one or more individuals from the second population based on the predicted phenotype data of the second population; and
h) developing one or more varieties from the selected one or more individuals, wherein the one or more varieties exhibit suitable phenotype for the second geographic area.

7. The method of any one of claims 1-6, wherein the individuals in the first population are hybrids and the individuals in the second population are inbred lines or hybrids that may or may not have parental inbred lines in common with the hybrids from the first population.

8. The method of any one of claims 1-6, wherein the individuals in the first population are inbred lines, breeding populations, or hybrids, and the individuals in the second population are segregating lines from breeding populations.

9. The method of any one of claims 1-6, wherein the individuals in the first population are parental lines and the individuals in the second population are filial lines derived from the parental lines.

10. The method of any one of claims 1 and 3-6, wherein the selection is for advancing the selected one or more individuals to a further stage in a breeding program.

11. The method of any one of claims 1 and 3-6, wherein the selection is for testing performance of the selected one or more individuals in a field.

12. The method of any one of claims 1 and 3-6, wherein the selected one or more individuals are segregating lines, inbred lines, or hybrid lines.

13. The method of any one of claims 1 and 3-12, wherein the selection is applied using a selection intensity.

14. The method of any one of claims 1 and 3-13, further comprising producing offspring from the selected one or more individuals.

15. The method of claim 14, wherein the offspring are produced by selfing, crossing, or asexual propagation.

16. The method of any one of claims 14-15, further comprising growing the offspring into maturity.

17. The method of any one of claims 1-16, wherein the first population is a training population and the second population is a prediction population.

18. The method of any one of claims 1-17, wherein the second population is a genetically diverse population.

19. The method of any one of claims 1-18, wherein the second population is a genetically uniform population.

20. The method of any one of claims 1-19, wherein the second population is an individual.

21. The method of any one of claims 1-20, wherein the first geographic area and the second geographic area are the same geographic area.

22. The method of any one of claims 1-21, wherein the second geographic area is a target breeding zone or a target market zone.

23. The method of any one of claims 1-22, wherein the envirotype data is time data, location data, weather data, soil data, companion organism data, management data, crop canopy data, cultivation area data, or a combination thereof.

24. The method of claim 23, wherein the time data is century, decade, year, season, month, day, hour, minute, second, or a combination thereof.

25. The method of claim 23, wherein the location data is latitude, longitude, altitude, or a combination thereof.

26. The method of claim 23, wherein the weather data is temperature, humidity, pressure, zonal wind speed, meridional wind speed, long-wave radiation, fraction of total precipitation that is convective, convective available potential energy, potential evaporation, precipitation hourly total, short-wave solar radiation, photoperiod, or a combination thereof.

27. The method of claim 23, wherein the soil data is soil type, soil structure, soil moisture, soil depth, soil organic matter content, soil density, soil pH, soil fertility, soil salinity, or a combination thereof.

28. The method of claim 23, wherein the companion organism data is soil fauna, insects, animals, weeds, or a combination thereof.

29. The method of claim 23, wherein the management data is intercropping management, covercropping management, rotating cropping management, or a combination thereof.

30. The method of claim 23, wherein the crop canopy data is obtained from an aerial platform.

31. The method of any one of claims 1-30, wherein the envirotype data is grouped according to the growth stages of the individuals.

32. The method of any one of claims 1-31, wherein the envirotype data is an envirotype map.

33. The method of any one of claims 1-32, wherein the one or more individuals are a crop selected from the group consisting of maize, soybean, wheat, sorghum, barley, oats, rice, millet, canola, cotton, cassava, cowpea, safflower, sesame, tobacco, flax, sunflower, a grain crop, a vegetable crop, an oil crop, a forage crop, an industrial crop, a woody crop, and a biomass crop.

34. The method of any one of claims 1-33, wherein the statistical model estimates the effects of genetic markers in interaction with the envirotype on the phenotype of the individuals of the first population.

35. The method of any one of claims 1-34, wherein the statistical model comprises a genotype variable, an envirotype covariate, and an interaction term between the genotype variable and the envirotype covariate.

36. The method of any one of claims 1-35, wherein the statistical model is a linear regression model, a logistic regression model, a Bayesian ridge regression model, a lasso regression model, an elastic net regression model, a decision tree model, a gradient boosted tree model, a neural network model, or a support vector machine model.

37. The method of any one of claims 1-36, wherein the predicted phenotype data of the second population are genomic estimated breeding values (GEBVs).

38. The method of any one of claims 1-37, wherein building the statistical model further comprises training the statistical model, tuning the statistical model, validating the statistical model, and/or updating the statistical model.

39. A variety developed by the method of claim 6.

40. A computer-implemented method for predicting phenotype data of a population in a geographic area for use in breeding, comprising:
a) receiving genotype data and envirotype data of a population of individuals in a geographic area; and
b) applying a statistical model to the genotype data and envirotype data of the population to obtain a prediction of phenotype data of the population in the geographic area,
wherein the statistical model is configured to receive genotype data and envirotype data of a population of individuals in a geographic area and output a prediction of phenotype data of the population in the geographic area; and
c) outputting the prediction of phenotype data of the population in the geographic area.

41. The method of claim 40, further comprising selecting one or more individuals from the population based on the predicted phenotype data of the population; and informing a user of the selected one or more individuals for breeding.

42. The method of any one of claims 40-41, wherein the statistical model is a trained model selected from the group consisting of linear regression model, a logistic regression model, a Bayesian ridge regression model, a lasso regression model, an elastic net regression model, a decision tree model, a gradient boosted tree model, a neural network model, and a support vector machine model.

43. A non-transitory computer-readable storage medium storing one or more programs for predicting phenotype data of a population in a geographic area for use in breeding, the one or more programs comprising instructions, which when executed by one or more processors of an electronic device having a display, cause the electronic device to:
a) receiving genotype data and envirotype data of a population of individuals in a geographic area; and
b) applying a statistical model to the genotype data and envirotype data of the population to obtain a prediction of phenotype data of the population in the geographic area,
wherein the statistical model is configured to receive genotype data and envirotype data of a population of individuals in a geographic area and output a prediction of phenotype data of the population in the geographic area; and
c) outputting the prediction of phenotype data of the population in the geographic area.

44. The computer-readable storage medium of claim 43, further comprising instructions for selecting one or more individuals from the population based on the predicted phenotype data of the population; and informing a user of the selected one or more individuals for breeding.

45. The computer-readable storage medium of any one of claims 43-44, wherein the statistical model is a trained model selected from the group consisting of linear regression model, a logistic regression model, a Bayesian ridge regression model, a lasso regression model, an elastic net regression model, a decision tree model, a gradient boosted tree model, a neural network model, and a support vector machine model.

46. The computer-readable storage medium of any one of claims 43-45, wherein the predicted phenotype data of the population are genomic estimated breeding values (GEBVs).

47. An electronic device for predicting phenotype data of a population in a geographic area for use in breeding, comprising:
a display;
one or more processors;
a memory ; and
one or more programs, wherein the one or more programs are stored in the memory and configured to be executed by the one or more processors, the one or more programs including instructions for:
a) receiving genotype data and envirotype data of a population of individuals in a geographic area; and
b) applying a statistical model to the genotype data and envirotype data of the population to obtain a prediction of phenotype data of the population in the geographic area,
wherein the statistical model is configured to receive genotype data and envirotype data of a population of individuals in a geographic area and output a prediction of phenotype data of the population in the geographic area; and
c) outputting the prediction of phenotype data of the population in the geographic area.

48. The system of claim 47, wherein the computer-readable storage medium further comprises instructions for selecting one or more individuals from the population based on the predicted phenotype data of the population; and informing a user of the selected one or more individuals for breeding.

49. The system of any one of claims 47-48, wherein the statistical model is a trained model selected from the group consisting of linear regression model, a logistic regression model, a Bayesian ridge regression model, a lasso regression model, an elastic net regression model, a decision tree model, a gradient boosted tree model, a neural network model, and a support vector machine model.

50. The system of any one of claims 47-49, wherein the predicted phenotype data of the population are genomic estimated breeding values (GEBVs).
